(19) United States
(12) Patent Application Publication
(10) Pub. No.: US 2012/0122162 A1
(43) Pub. Date: May 17, 2012

(54) ENZYMATIC HYDROLYSIS OF PRE-TREATED BIOMASS

(75) Inventors: Rodolfo ROMERO, Clifton Park, NY (US); Bertil Stromberg, Diamond Point, NY (US)

(73) Assignee: Andritz Inc., Glens Falls, NY (US)

(21) Appl. No.: 13/295,331

(22) Filed: Nov. 14, 2011

Related U.S. Application Data
(60) Provisional application No. 61/419,519, filed on Dec. 3, 2010.
(60) Provisional application No. 61/413,777, filed on Nov. 15, 2010.

(51) Int. Cl. C12P 19/14; C12P 1/04; C12P 1/02; C12P 1/00

(52) U.S. Cl. 435/99; 435/41; 435/170; 435/171

(57) ABSTRACT

This disclosure relates to a method for performing high solids saccharification comprising by (a) providing a cellulosic biomass; (b) pretreating the cellulosic biomass in a pretreatment process to produce a pretreated cellulosic biomass; (c) adjusting said pretreated cellulosic biomass to a solids concentration of 6% to 35% w/w and a starting pH of between 5-7; and (d) hydrolyzing the pretreated biomass with at least one aqueous hydrolyzing liquid comprising at least one enzyme selected from the group consisting of a cellulase, a saccharification enzyme, and a combination thereof for a period of time, to hydrolyze at least a part of the pretreated cellulosic biomass to a cellulosic hydrolysate, said cellulosic hydrolysate comprising fermentable sugars.

### CROSS RELATED APPLICATIONS

This application claims the benefit of U.S. Provisional Patent Application 61/413,777, filed Nov. 15, 2010 and U.S. Provisional Patent Application 61/419,519, filed Dec. 3, 2010, the entirety of each application is incorporated by reference.

### BACKGROUND OF THE INVENTION

A high enzymatic hydrolysis yield and a higher yield using high consistency biomass (i.e., high solids content as measured w/w) under enzymatic saccharification has been a long felt need in the industry because it would reduce cost and improve efficiency. However, despite many years and many attempts to optimize the process by many in the industry, yields have not improved to a level that is completely satisfactory.

### SUMMARY OF THE INVENTION

The understanding of enzymatic saccharification of pretreated lignocellulosic material is of great importance. There are several important commercially available enzymes in the market that are used for this purpose. The conditions of pH and temperature performance of any particular enzyme are very well defined and it is clearly indicated by its manufacturer and it depends on the type of enzyme or enzymes in the complex pool. Commercial cellulases work best at pH around 4.8-5.0 (1, 2, 3). In this study it was found that, contrary to current understanding, optimum pH of cellulases is unexpectedly different than that recommended by its manufacturer at higher solids loads saccharification. The optimum pH changes depending on the consistency or solids loads of the matrix where the enzyme is acting upon in a way that is not previously disclosed. As a representative biomass, steam exploded corn Stover was tested with cellulases and xylanases at different pH, consistencies and ionic strength with the expectation of confirming the current optimal pH of around 4.8-5.0. Results showed that the optimum pH at lower consistency (1% w/w) is the same as the one that is recommended by manufacturer and the literature; however at higher consistency the value obtained was higher (pH 5.5 to pH 6.5) instead of the pH 4.8. The difference could represent of up to 30-50% higher yields and hence of great importance for the economics of any second generation fuel production. An explanation of this behavior could be associated with the Donnan effect theory. This effect indicates that the presence of charge groups in the fiber matrix creates a pH gradient within the slurry. If the charged groups are negatively charged this would create a local or internal pH lower than the surrounding liquid pH. This could explain why by reducing the concentration of H⁺ higher enzymatic yields were observed (4). The suspected Donnan effect on the pre-treated material is surprising, as chemical pulps subjected to the same enzymatic treatments did not exhibit the same shift in optimum pH when the solids loading was increased.

It is an aim of some aspects of the present disclosure to provide improved process conditions to increase the effectiveness of cellulases during saccharification of lignocellulosic material.

Further, it is an aim of some aspects of the present disclosure to provide an improved system for the production of target chemicals, such as ethanol, butanol, acetic acid, butyric acid and others, from cellulosic biomass.

Still further, it is an aim of some aspects of the present disclosure to provide for improvements on saccharification of enzymatic hydrolysis at high solids loading (≧10% w/w) of biomass during the production of the target chemical not disclosed before.

Moreover, it is an aim of some aspects of the present disclosure to provide for a range of pH where enzyme would out-perform the well-established pH optimum of cellulases during high solids saccharification of lignocellulosic material.

In addition, it is an aim of some aspects of the present disclosure to further provide an improvement of enzymatic saccharification by means of allowing a natural change of pH to lower values caused by the liberation of hydrogen proton into the media.

One embodiment of the invention relates to a method for performing high solids saccharification comprising the following steps: (a) providing a cellulosic biomass (also referred to as the feedstock); (b) pretreating the cellulosic biomass in a pretreatment process to produce a pretreated cellulosic biomass; (c) adjusting said pretreated cellulosic biomass to a solids concentration of 6% to 35% w/w and a starting pH of between 5-7; (d) hydrolyzing the pretreated biomass with at least one aqueous hydrolyzing liquid comprising at least one enzyme selected from the group consisting of a cellulase, a saccharification enzyme, and a combination thereof for a period of time, to hydrolyze at least a part of the pretreated cellulosic biomass to a cellulosic hydrolysate, said cellulosic hydrolysate comprising one or more fermentable sugars.

In the method, the pretreating step may comprise contacting the cellulosic biomass with at least one aqueous pretreatment fluid to produce a pretreated cellulosic biomass. In one embodiment, the pretreating step may be conventional steam explosion. Steam explosion usually involves the thermal treatment of biomass with water under pressure. Then the pressure is suddenly released, causing the biomass to break and explode.

In conventional steam explosion, steam under high pressure penetrates the lignocellulosic structures by diffusion. The steam condenses under the high pressure thereby “wetting” the material. The “wet” biomass is “exploded” when the pressure within the reactor is released. Typically, the material is driven out of the reactor through a small nozzle by the induced force. Several phenomena occur at this point. First, the condensed moisture within the structure evaporates instantaneously due to the sudden decrease in pressure. The expansion of the water vapor exerts a shear force on the surrounding structure. If this shear force is high enough, the vapor will cause the mechanical breakdown of the lignocellulosic structure.

In another embodiment, the pretreating step may comprise pretreating the cellulosic biomass by Advanced Steam Explosion. Advanced Steam Explosion may comprises the steps of: (b1) pretreating the cellulosic biomass in a first pressurized reactor, wherein the cellulosic biomass undergoes hydrolysis in the first pressurized reactor; (b2) discharging the cellulosic biomass from the first pressurized reactor to a pressurized sealed device having a first pressurized coupling to a cellulosic biomass discharge port of the first pressurized reactor; (b3) maintaining a vapor phase in the first pressurized reactor by injecting steam into the first pressurized reactor, wherein the injected steam provides heat energy to the cellulosic biomass in the first pressurized reactor; (b4) washing the cellulosic biomass in a downstream region of the first pressurized reactor or the pressurized sealed device; (b5) draining a liquid including dissolved hemi-cellulosic material extracted from the cellulosic biomass from at least one of the first pressurized reactor and the pressurized sealed device; (b6) discharging the cellulosic biomass from the pressurized sealed device through a second pressurized coupling to a second pressurized reactor, wherein the cellulosic biomass is maintained at a higher pressure in the second pressurized reactor than in the first pressurized reactor; (b7) in the second pressurized reactor, infusing cells of the cellulosic biomass with steam or water vapor by injecting steam or water vapor into the second pressurized reactor, and (b8) rapidly releasing a pressure applied to the cellulosic biomass infused with water to cause steam expansion in the cells of the cellulosic biomass and refine the cellulosic biomass to produce a pretreated cellulosic biomass. The pressurized sealed device is also referred to as the pressurized sealing device.

The method produces, inter alia, one or more fermentable sugars. Another embodiment of the methods involves an additional step of fermenting the produced one or more fermentable sugars in a fermentation process utilizing at least one microorganism. The microorganism may be, for example, at least one microorganism is selected from the group consisting of wild type bacteria, recombinant bacteria, wild type filamentous fungi, recombinant filamentous fungi, wild type yeast, recombinant yeast, and a combination thereof.

The pretreated cellulosic biomass of the methods of this disclosure can be adjusted for solids concentration by the addition or removal of solvent (including water) or the addition or removal of solids. Examples of the lower limit of solids concentration (w/w) can be ≧6%, ≧10%, 12%, ≧18% or ≧20%. Examples of the upper limit on solid concentration (w/w) can be ≦35%, ≦25%, ≦23% or ≦20%. The upper limits and lower limits of solids concentration may be combined in any fashion. For example, the pretreated cellulosic biomass may have a solids concentration of 10% to 35% w/w, 10% to 20% w/w, 15% to 35% w/w, or 35% to 20% w/w.

In one embodiment, the starting pH of the pretreated biomass is between 5.5-6.5, such as between pH 5.7 to pH 6.1. These pH ranges may be maintained throughout steps (c) and (d). Alternatively, the pH may be set once in the beginning (i.e., step (c)) and not be adjusted through the rest of the reaction in step (d). In another embodiment, the starting pH is maintained between pH 5.7 to pH 6.1 in steps (c) and the pH is decreased in step (d) to a pH of 5.1 to 5.5 over the duration of step (d). For example, the ending pH can be from pH 5.2 to 5.4, such as, for example pH 5.3. pH adjustment is well known. It can be made, for example, by adding acids or bases to a reaction. The acid and base do not have to be pure products but can also be byproducts, liquors, fluids including waste fluids and waste solids from other related or unrelated reactions. The acid or base can also be additional starting material or product material. Maintaining or establishing a pH may involve, for example, extracting a sample once or periodically, determining the pH in the sample, and adding the appropriate pH adjusting material as described above. This procedure may be repeated every few hours (12, 6, 3, 2 or 1 hour) hourly or more frequently. In the embodiment described above, the pH is linearly decreased over the period of step (d). Step (d) (that is, the period or duration of step (d)) may be between 12 to 200 hours long. For example, a lower limit to the prior or duration of step (d) may be 12 hours, 24 hours, 36 hours, 50 hours, 75 hours or 100 hours. An upper limit to step (d) may be 100 hours, 125 hours, 150 hours, 175 hours or 200 hours. Any combination of lower and upper limits in for step (d) may be combined.

The methods of the disclosure can achieve surprising results over the conventional method of saccharification. For example, the method can achieve a saccharification of the pretreated cellulosic biomass of over 50%, over 55%, over 60%, over 65%, over 70% or over 75%.

The feedstock for the methods may be any cellulosic biomass. The biomass may be any lignocellulosic material or may be a mixture that comprises a lignocellulosic material (e.g., a byproduct of a (industrial) process or a mixed waste product). Lignocellulosic material refers to a material that comprises (1) cellulose, hemicellulose, or a combination and (2) lignin. Throughout this disclosure, it is understood that cellulose may refer to cellulose (i.e., cellulose only), hemicellulose, or a combination thereof.

Examples of a biomass or lignocellulosic material that can be treated with the methods of the disclosure include, at least, materials comprising corn stovers, bioenergy crops, agricultural residues, municipal solid waste, industrial solid waste, yard waste, wood and forestry waste, sugar cane, switchgrass, wheat straw, hay, barley, barley straw, rice straw, grasses, waste paper, sludge from paper manufacture, corn grain, corn cobs, corn husks, grasses, wheat, wheat straw, hay, rice straw, sugar cane bagasse, sorghum, soy, trees, branches, wood chips and sawdust.

The term “cellulase enzymes” or “cellulase” refer to enzymes that catalyze the hydrolysis of cellulose to products such as glucose, cellobiose, and other cellooligosaccharides. Cellulase may refer to cellulase, hemi-cellulase, or a combination thereof and can be a multienzyme mixture, produced by a number of microorganisms, comprising exo-cellobiohydrolases (CBH), endoglucanases (EG) and β-glucosidases. Among, the most widely studied, characterized, and commercially produced cellulases are those obtained from fungi of the genera Aspergillus, Humicola, and Trichoderma, and from the bacteria of the genera Bacillus and Thermobifida.

The term saccharification enzyme refers to one or more enzymes that aids in the process of breaking down a complex carbohydrate (e.g., starch and/or cellulose) into its monosaccharide components. The enzymes may be a mixture of one or more of the following: endoglucanases, exoglucanases, cellobiohydrolases, β-glucosidases, xylanases, endoxylanases, exoxylanases, β-xylosidases, arabinoxylanases, mannases, galactases, pectinases, glucuronidases, amylases, α-amylases, β-amylases, glucoamylases, α-glucosidases, and isoamylases.

The methods of the invention may be used to produce one or more (a plurality) primary target chemicals following the saccharification step and following fermentation. The primary target chemical can be an alcohol. Examples of the primary target chemical that can be used include methanol, ethanol, butanol, acetic acid, butyric acid and a combination thereof.

Experimental data which supports these various embodiments are listed throughout this specification. One supporting experiment is listed immediately below in FIG. 1.

FIG. 1 depicts the effect of pH during enzymatic hydrolysis of high consistency pretreated corn stover. Specifically in this figure is shown the effect controlling at constant pH versus adjusting pH just at the beginning of saccharification and letting the process (reaction) follow its natural course. This result indicates that the Donnan effect is still playing an important role on creating a pH gradient on the media during the reaction. However, as we are the first to show, as the reaction proceeds fibers are being broken down by cellulases causing the pH optima to go back in direction of original optimum values. As can be seen, the difference in yield is considerable and we have achieved a significant increase in process efficiency.

### BRIEF DESCRIPTION OF THE DRAWINGS

FIG. 1 depicts high consistency enzymatic hydrolysis of PCS with different control pH patterns.

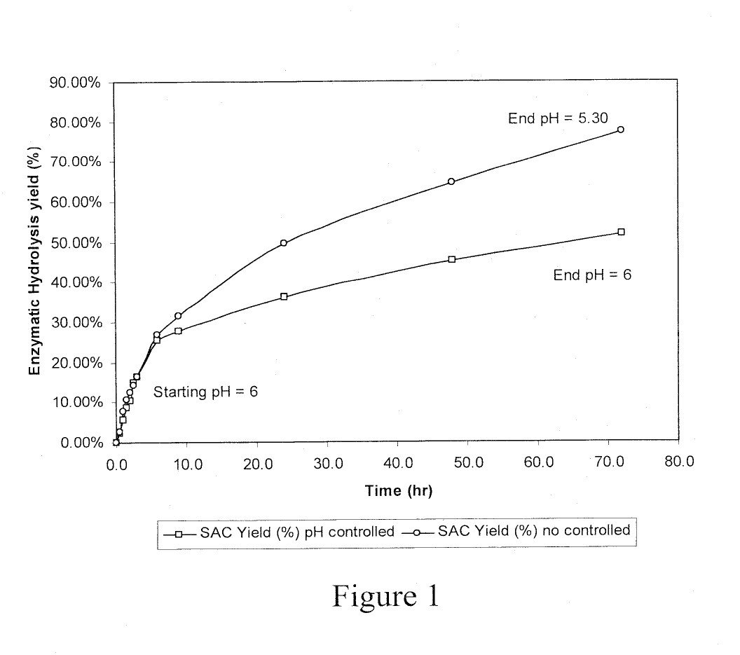

FIG. 2 depicts an advance steam explosion concept from Andritz. In the figure, SAC denotes Saccharification; SRP denotes Standard reference pulp; PCS denotes Pretreated corn stover; PITC denotes Andritz's Pruyn's Island Technical Center; and ASE denotes Advance Steam Explosion.

FIG. 3 depicts a graph showing enzymatic saccharification of kraft pulp and PCS using low and high consistency in rotisserie shaker incubator. The graph shows the conversion efficiency from cellulose to glucose using enzymatic hydrolysis of pulp and PCS using low and high consistency. Results are after 168 hours of reaction time.

FIG. 4 depicts a comparison of type of mixing effect on enzymatic hydrolysis of kraft pulp and PCS. The graph shows the effect of type of agitation on enzymatic saccharification of pulp and PCS at lower consistency. Results are after 168 hours of reaction time.

FIG. 5 depicts effect of the type of shaker incubator on SAC of SRP and PCS. The graph shows the effects of mixing in saccharification of pulp and steam exploded corn stover combined. Results are after 168 hours of reaction time.

FIG. 6 depicts the final enzymatic saccharification yield (%) at high consistency of acid and kraft pulps. The graph shows enzymatic hydrolysis comparison between acid and kraft pulps at 10% consistency. There are two groups of columns with two columns each. Within each group, the left column represents yield after 72 hours treatment (Yield72(%)) and the right column represents yield after 168 hours treatment (Yield168(%)).

FIG. 7 depicts final enzymatic saccharification yield (%) at low consistency of acid and kraft pulps. The graph shows enzymatic hydrolysis comparison between acid and kraft pulps at 2% consistency. There are two groups of columns with two columns each. Within each group, the left column represents yield after 72 hours treatment (Yield72(%)) and the right column represents yield after 168 hours treatment (Yield168(%)).

FIG. 8 depicts enzymatic saccharification yield (%) at 168 hours of acid and kraft pulp at low and high consistencies. The graph shows enzymatic hydrolysis comparison between acid and kraft pulps at 2% (LC) and 10% (HC) consistency. There are two groups of columns with two columns each. Within each group, the left column represents HC sac (High consistency saccharification yield) and the right column represents LC sac (Low consistency saccharification yield).

FIG. 9 depicts enzymatic hydrolysis of kraft pulp at different pH and at low and high consistency. The graph shows the effect of consistency on cellulases optimum pH during saccharification of kraft pulp at low (LC) and high (HC) consistencies. Yield (%) LC is represented by the purple line with square markers. Yield (%) HC is represented by the blue line with diamond markers. Reaction time 168 hours.

FIG. 10 depicts the effect of solids loading on optimum cellulase pH of enzymatic hydrolysis of steam exploded corn stover. The graph shows the effect of consistency on cellulases optimum pH during saccharification of steam exploded corn stover (ASE H+35/170; 2/195). Low consistency is represented by the dark blue line with diamond markers. High consistency is represented by the purple line with square markers. Reaction time 168 hours.

FIG. 11 depicts the effect of solid loads on enzymatic saccharification optimum pH of steam exploded energy cane (ASE H+30/150; 2/200). The graph shows the effects of saccharification consistency on optimum pH of steam exploded energy cane (ASE H+0.95%+30/150; 2/200). Low consistency is represented by the dark blue line with diamond markers. High consistency is represented by the purple line with square markers. Reaction time 168 hours.

FIG. 12 depicts the effect of enzymatic hydrolysis of kraft pulp at different pH and at low and high consistency. The graph shows the effect of increased ionic strength on cellulases optimum pH during saccharification of kraft pulp for 168 hours at low and high consistencies. Yield (%) LC is represented by the purple line marked with squares. Yield (%) HC is represented by the blue line marked with diamonds. HC CaOH2 0.05 M is represented by the yellow line marked with triangles. HC CaOH2 0.1 M is represented by the light blue line marked with “X”s.

FIG. 13 depicts the effect of ionic strength on pH optima of HC enzymatic hydrolysis of advanced steam explosion corn stover. The graph shows the effect of increased ionic strength on cellulases optimum pH during saccharification for 168 hours of pretreated corn stover at high consistencies. The line marked with blue diamonds represents yield (%) as is. The line marked by purple squares represents yield (%) CaOH₂ 0.03M. The line marked by yellow triangles represents yield (%) LiCl 0.01 M. The line marked by blue “X”s represents yield (%) KCl 0.01 M. The line marked by a dark purple “*” represents CaCl₂ 0.05M.

FIG. 14 depicts enzymatic saccharification yield of solvent extracted steam exploded corn stover at low and high consistencies. The chart shows high consistency enzymatic saccharification at 168 hours of solvent extracted pretreated corn stover. There are four groups of columns with two columns each. Within each group, the left column represents LC saccharification and the right column represents HC saccharification.

### DETAILED DISCUSSION

PITC (Andritz's Pruyn's Island Technical Center) enzymatic evaluations of different pretreated materials have shown that high consistency saccharification perform differently than low consistency enzymatic hydrolysis. In fact a reduction of about 30% in saccharification is observed in the former. One of the explanations is based on the fact that at high consistency end product inhibition would reduce the effectiveness of the enzymes (1). Others indicated the presence of higher concentration of degradation products that are liquefied and hence posing an inhibitory effect on the enzyme performance at higher consistencies (1). To eliminate end product inhibition during experimentation is use low consistency enzymatic hydrolysis (1). Another way to eliminate this inhibition is by the use of SSF (simultaneous saccharification and fermentation) where this is reduced considerably by concomitant consumption of end product by yeast (in the case of glucose). Large scale low consistency conditions would rarely be used as it would be prohibitive by the economics. SSF requires the use of live yeast or any other microorganism to reduce glucose which causes end product inhibition and so the saccharification enzymes would not be inhibited. However, when using live yeast, other sets of conditions are required which are not the optimum for enzyme saccharification. Such conditions are lower temperature and different pH. Toxicity is another factor which would reduce the effectiveness of the use of yeast. This also would include other variables that would mask the actual ideal condition for enzyme study on the cellulose/lignin/hemicellulose matrix and understanding of its kinetics and inhibitions (6). The real understanding of enzyme action at higher consistency is then of great importance.

The type of the metal counter-ions in the carboxylic groups and their dissociation has been proposed to play an important role in enzymatic pulp treatment, (4). The removal of the metal cations from the pulps by EDTA or by acid washing prior to the enzymatic treatment has rendered the pulp practically non-hydrolysable by Trichoderma reesei Xylanases (4). According with the Donnan theory, the acidic groups bound to the fiber matrix induce a pH gradient between the fiber wall and the outer solution (5). The magnitude of the pH difference depends on the content of acidic groups, the type of counter ions of the acidic groups and also the content and type of ions in the solution. Increase in the ionic strength of the solution diminished the effect, and at a certain electrolyte level the phenomenon is no longer observed (5). As the pH of pulp suspensions for various chemical as well as enzymatic treatments is adjusted by measuring the pH of the bulk solution, the actual pH at the reactive site in the fiber wall is influenced by the ionic state of the system. This is of practical importance as the purity and thus the ionic strength of the process water may vary, depending on the type of application.

Our work has shown that different enzymatic hydrolysis yields results are obtained when using low and high consistency experiment when using kraft pulp. High consistency enzymatic saccharification is actually higher than lower consistency which is totally in contrary to the state of art and contrary to current theory and teachings. Current theories and practice has generally agree that lower consistency biomass should produce better yields (e.g., a percent of starting materials converted) at least because there are less inhibitor buildup with lower consistency material. In light of current teachings, our results are surprising and unexpected. In fact, because our results are surprising and unexpected, it has not been practiced or suggested by any of the current literature.

One possible explanation, after we considered our results and can view the available theories in hindsight, is the Donnan effect. A Donnan effect has never been proposed for this type of chemical process. In the following study, the Donnan effect, i.e., metal cation, ionic strength and thus the intrinsic pH on the enzymatic action was investigated using advance steam explosion corn stover as a model substrate. A set of experiments were designed to elucidate and understand the mechanism of action of enzyme in presence advance steam exploded corn stover at different pH and ionic strength.

Therefore, one objective of this study is to find a more cost effective method of enzymatic hydrolysis of biomass. The investigation involves determining whether the Donnan effect could explain why complete saccharification at the recommended pH using commercial cellulases can not be achieved when using higher consistency but yet possible at lower consistency experiments.

### Material and Methods:

Standard reference pulp (SRP) and steam exploded corn stover (PCS) were used as the only non-steam exploded and steam exploded lignocellulosic material respectively. Standard reference pulp from PAPRICAN analytical service 570 boul. St-Jean Pointe-Claire QC, Canada H9R 3J9 was chosen due to that it has been used extensively in PITC as analytical lignocellulosic reference material. Corn stover from Iowa collected from the second crop of 2008 was prepared by the process of advance steam explosion (ASE) from Andritz. The conditions were the following. Corn stover was size reduced using a Troy-Bilt CS 4265 Chipper shredder. The chipped corn stover was presoaked with 0.5% w/w solution of H₂SO₄ for 2 hours at 35° C. at L/S ratio of 10:1. After this period the material was pressed in industrial press at 1000 psig. Pressed material was fluffed by hand. A steam gun manufactured by Andritz Inc was charged with 4000 g oven dry weight (OD). The equipment was set to hydrolyze between 30 minutes and 60 minutes at range temperature of 150° C. to 180° C. This material was then pressed again to remove C₅ sugars in liquid form and manually fluffed again. The same steam gun was charged with the processed material and steam exploded for 2-4 minutes at 195° C. to 205° C. Product was collected and store at 4° C. for further analysis.

NREL low solid saccharification protocol (NREL/TP 510-42630) was used throughout the experiments. High solids experiments were carried out using PITC protocol BIO-2010-001. Cellic C-tech cellulases enzyme complex from Novozyme at 7% w/w C₆. Cellic H-tech xylanases enzyme complex from Novozyme at 0.5% (w/w). Instruments from YSI Life Sciences (glucose and other hydrocarbon monitors such as the YSI 2700 biochemistry analyzer) were used for glucose and xylose analysis.

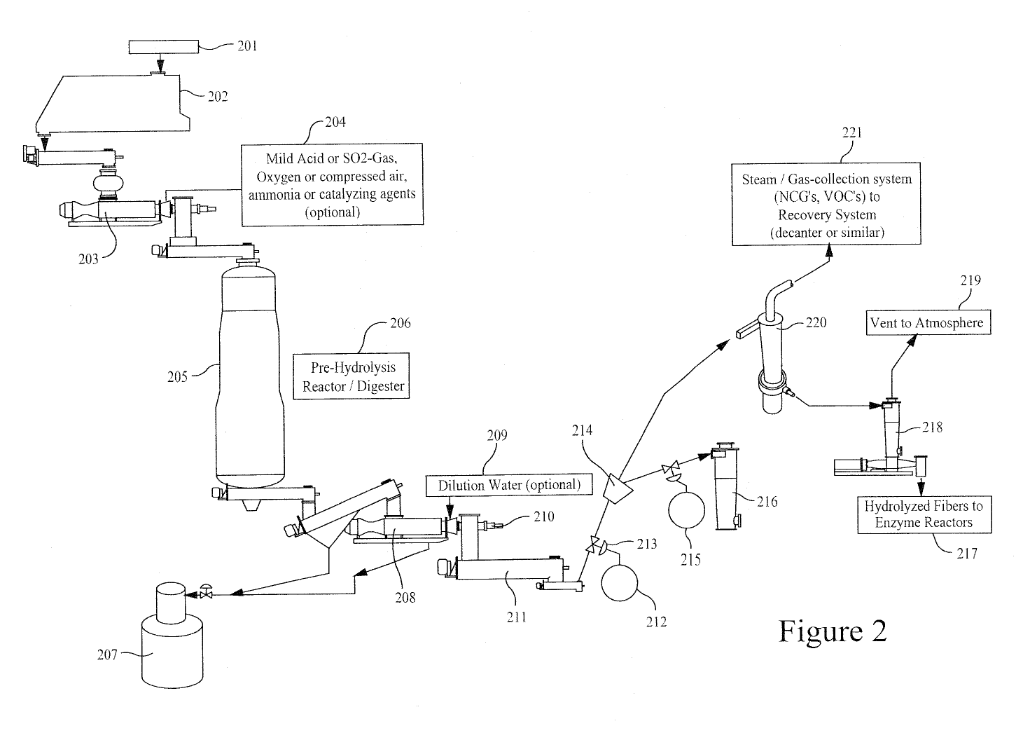

Advance steam explosion is a concept developed by Andritz that rely on the fact that removing hemicellulose prior steam explosion not only removes C5 sugars in a gentler way before steam explosion but also it was been demonstrated that removal hemicellulose improves enzymatic hydrolysis of the steam exploded holo-cellulose. A schematic of the concept is shown in FIG. 2. See, U.S. application Ser. No. 12/389,020 filed Feb. 19, 2009 and published as U.S. Publication No. 2009/0221814 which is incorporated herein by referenced in its entirety. One method of Advanced Steam Explosion would involve, for example, obtaining biomass feedstock and (a) pretreating the feed stock (e.g., cellulosic biomass) in a first pressurized reactor, wherein the feed stock undergoes hydrolysis in the first pressurized reactor; (b) discharging the feed stock from the first pressurized reactor to a pressurized sealed device having a first pressurized coupling to a feedstock discharge port of the first pressurized reactor; (c) maintaining a vapor phase in the first pressurized reactor by injecting steam into the first pressurized reactor, wherein the injected steam provides heat energy to the feedstock in the first pressurized reactor; (d) washing the feed stock in a downstream region of the first pressurized reactor or the pressurized sealing device; (e) draining a liquid including dissolved hemi-cellulosic material extracted from the feed stock from at least one of the first pressurized reactor and the pressurized sealed device; (f) discharging the feed stock from the pressurized sealed device through a second pressurized coupling to a second pressurized reactor, wherein the feed stock is maintained at a higher pressure in the second pressurized reactor than in the first pressurized reactor; (g) in the second pressurized reactor, infusing cells of the feed stock with steam or water vapor by injecting steam or water vapor into the second pressurized reactor, and (h) rapidly releasing a pressure applied to the feed stock infused with water to cause steam expansion in the cells of the feed stock and refine the feed stock. The product of this Advanced Steam Explosion process may be used as the pretreated cellulosic biomass of the process described in this disclosure. The method of claim 17 further comprising introducing at least one of mild acid, sulfur dioxide gas (SO₂), oxygen, compressed air, steam, water and catalyzing agents to the feed stock in at least one of the first pressurized reactor and the second pressurized reactor. In the ASE process, the step of pretreating the feed stock occurs in the first pressurized reactor having an internal temperature in a range of 110-160° C. or 110-175° C., a pressure in a range of 150-600 kilopascals or 150-1000 kilopascals, and wherein the feed stock remains in the first pressurized reactor for a about 10-60 minutes. Further, it is preferred that one or more of the following conditions are met (1) the feed stock flows as a continuous stream through the first pressurized reactor, the pressurized sealed device, the second pressurized reactor and to the rapid release of pressure downstream of the second pressurized reactor; (2) the rapid release of pressure reduces the pressure of the feed stock by at least ten bar; (3) the washing step is between said discharging step and draining step and washes the dissolved hemi-cellulosic material from said feedstock between said first pressurized reactor and said second pressurized reactor; (4) the washing step is performed at a temperature below 160° C. or below 140° C. Please note that the pressurized sealed device can also be a pressurized sealing device.

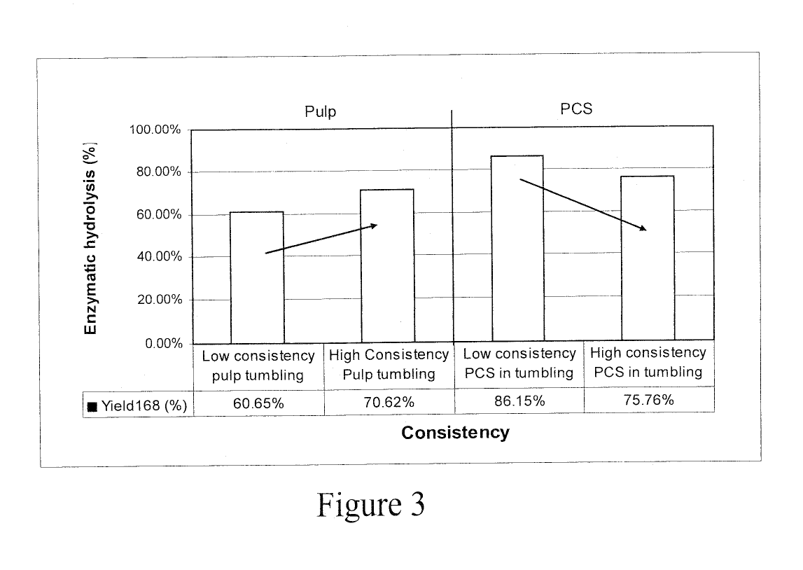

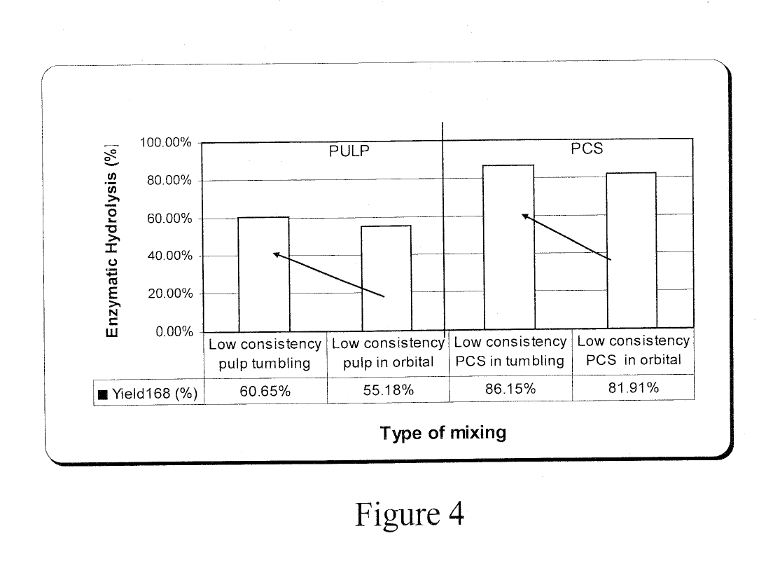

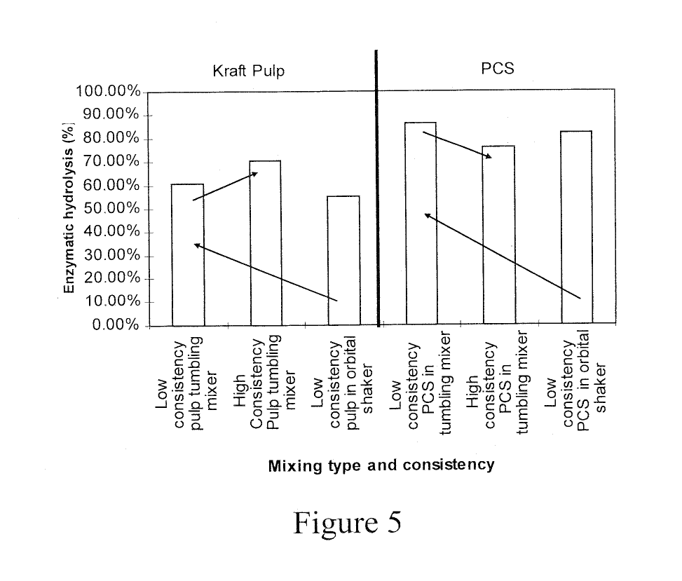

As previously mentioned and shown in FIG. 3, it is clearly observed the different results between enzymatic saccharification for 168 hours of pulp or PCS at low and high consistency conditions. We observe that the yield of low consistency pulp saccharification is lower than its correspondent high consistency. On the contrary the same analysis show opposite results when steam exploded corn stover is used. Conventional theory and practice would suggest that perhaps agitation difference between the two methods (normally low consistency is performed in orbital shaker whereas high consistency is performed in tumbling mixer) is affecting the results, if this would be the case theoretically in both cases (both high and low consistencies) with a better mixing, saccharification should improve. This only happens with low consistency experiment as shown in FIG. 4; however that is not what is happening when it is run side by side low and high consistency saccharification using same method. In FIG. 4 it is shown results from the two different agitation methods, the tumbling shaker and the orbital shaker. It is clear that the type of agitation (orbital or tumbling) when using low consistency experiment are better on the tumbling mixer which is the logic trend of better mixing producing better saccharification. However when high consistency is used again opposite results is obtained between pulp and steam exploded corn stover which is shown in a combine graph (FIG. 5).

This is observed over and over as the reference pulp is used in all experiment performed at PITC. This behavior leads us to theorize that other phenomena rather than mixing or inhibition could be controlling enzymatic saccharification and that it is depended on the type of matrix and its interaction with charge groups.

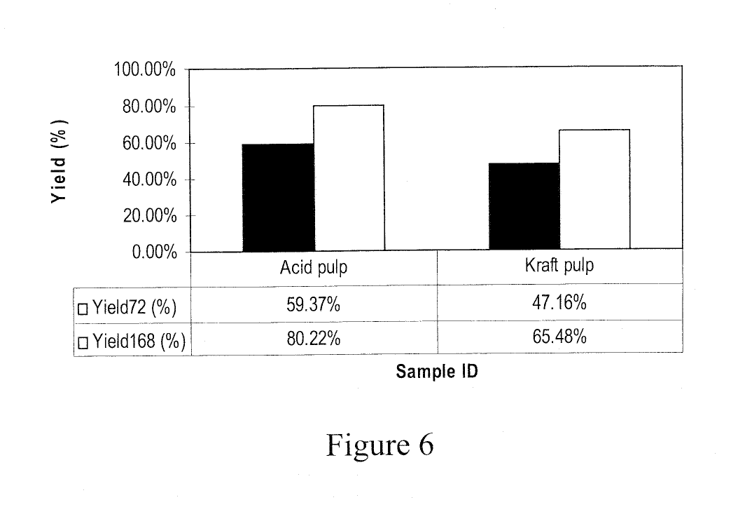

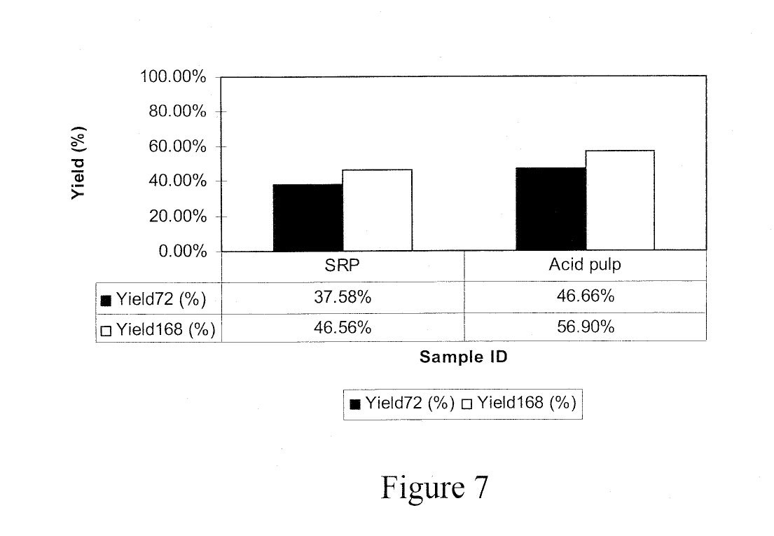

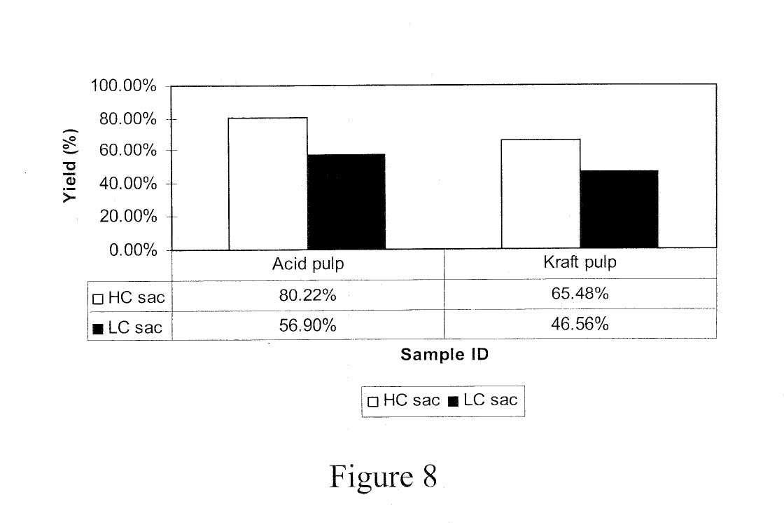

One possibility could be that the cellulases charge groups net balance (negative or positive) be more intensively repelled in steam exploded material as compared to kraft pulp, for example. To prove this possibility pulp produced by an acidic sulfite process was used for saccharification. Acid produced pulp in theory would have fewer metals as shown in table 1. A sample of an acid produced pulp was collected from Finch Paper and submitted to high and low consistency enzymatic saccharification. The low and high consistency experiment showed that the acid treated pulp performs similar or even better than the kraft pulp as shown on FIGS. 6, 7 and 8. This indicates that metal presence or absence themselves do not induce a similar response as the results shown on PCS. However, metal concentration in the acid wash pulp was much lower than the kraft pulp and better yield was observed in the earlier. Another difference between the two pulps is that the acid produced pulp was made from a mixture of hardwood and softwood, whereas the kraft pulp was made only from softwood.

TABLE 1
Metal content on pulps and PCS

| | | | | | PCS ASE |
|---|---|---|---|---|---|
| | | | | PCS ASE | H + 35/170 |
| | Finch | Softwood | PCS ASE | H + 35/170 | 2/195 |
| Description | pulp | SRP brown | H + 35/170; | 2/195 | Wash |
| (mg/Kg) | (mg/kg) | stock | 2/195 | CaDetox | Control |
| Al | 4.71 | 26.4 | 455 | 488 | 454 |
| Ba | <1 | <1.0 | 22.6 | 20 | 7.92 |
| Be | <1 | <1.0 | <1.0 | <1.0 | <1.0 |
| Ca | 725 | 1910 | 782 | 2130 | 320 |
| Co | <1 | <1.0 | <1.0 | <1.0 | <1.0 |
| Cr | <1 | <1.0 | 13.9 | 17.3 | 14.5 |
| Cu | <1 | <1.0 | 7.1 | 7.58 | 7.03 |
| Fe | <1 | 40.3 | 1660 | 1150 | 1330 |
| K | 30.6 | 33.3 | 383 | 76.7 | 82.6 |
| Mg | 139 | 360 | 295 | 147 | 121 |
| Mn | <1 | 58.5 | 10.1 | 6.29 | 4.89 |
| Na | 164 | 300 | 81.2 | 71.4 | 65.4 |
| P | 14.8 | 19.7 | 229 | 36.2 | 31.1 |
| S | 131 | 307 | 351 | 245 | 22.7 |
| Si | 14.5 | 131 | 72.3 | 52.4 | 43.4 |
| Sr | <1 | <1.0 | <1.0 | <1.0 | <1.0 |
| Zn | <1 | 11.1 | 7.47 | 8.48 | <1.0 |

Another possibility is that if the cellulases themselves are not significantly influenced by the charge groups then would the counter ion-charge group interactions in the matrix itself influence cellulases optimum pH. To determine if this possibility is correct, a set of different experiments were carried out with kraft pulp (SRP) as well as with steam exploded corn stover at different pH and different consistencies.

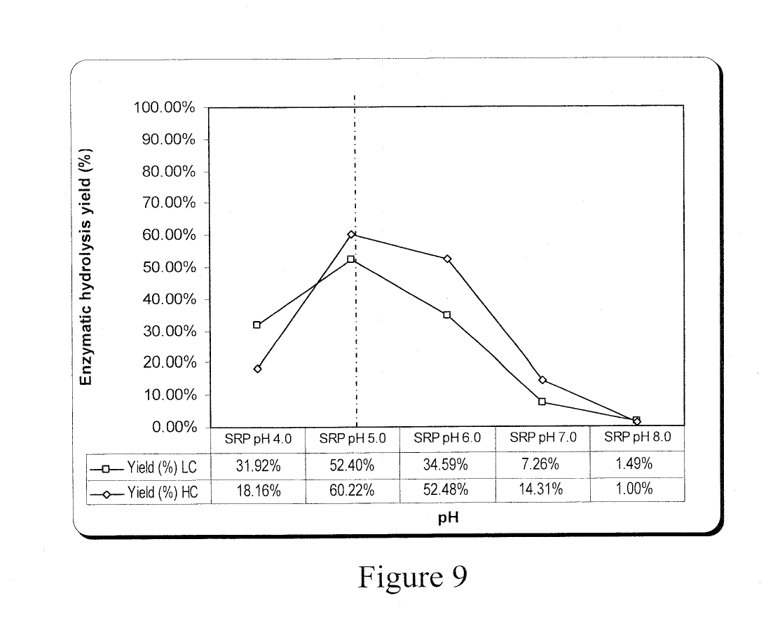

The enzyme optimum pH observed during saccharification of kraft pulp is in agreement with enzyme manufacturer recommended pH as shown in FIG. 9. An optimum pH of 5 is observed in both high and low consistency experiment. This is surprising, as Buchert (5) observed a shift in optimum pH at different consistencies when using xylanases on kraft pulp.

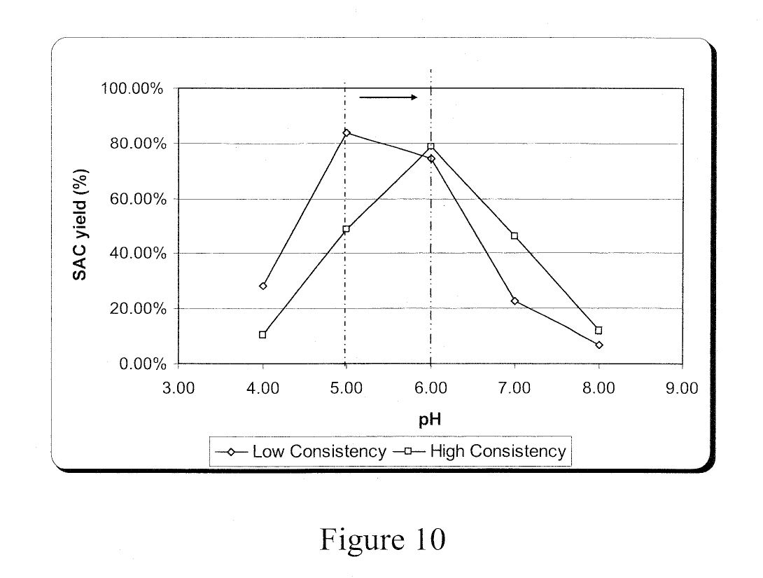

On the contrary when the same sets of experiments were run with steam exploded corn Stover, the opposite occurs. Observe the optimum pH switch between pH 5 at low consistency and pH 6 at higher consistency (FIG. 10). There is change of optimum pH when using higher consistency as compared to lower consistency experiment. The yield difference between pH 5 (50%) and pH 6 (82%) is in the order of 64% which is large in term of economics.

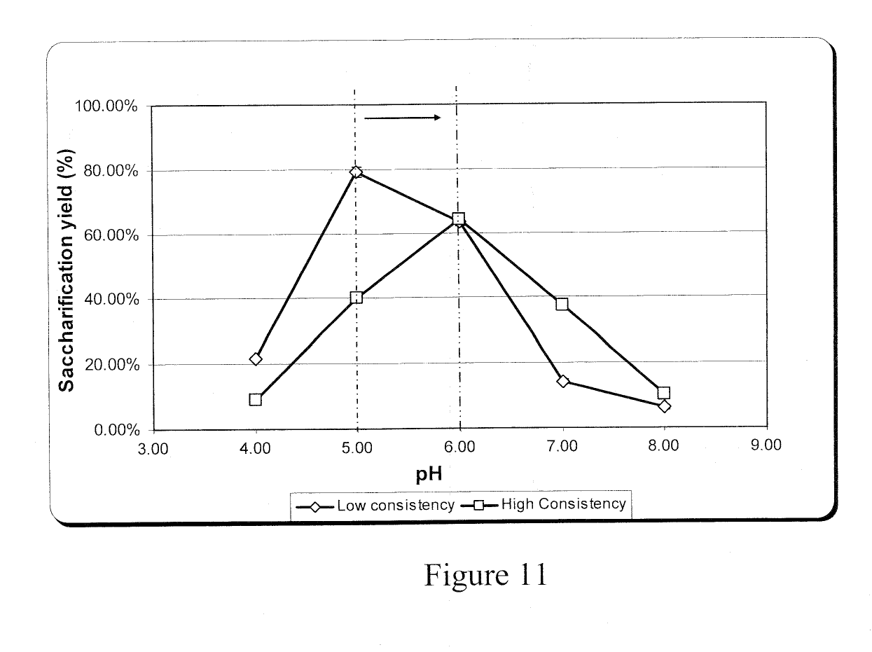

This holds true not only for corn stover but also to steam exploded energy cane as observed in FIG. 11. In this case an increase of 55% in yield is achieved just by performing saccharification at pH 6 instead of pH 5.

Although the same level of saccharification obtained using lower consistencies is not reached, a great improvement is achieved.

One possible explanation to this phenomenon is by the use of the Donnan theory. According with this theory the acidic groups bound to the fiber matrix induces a pH gradient between the fiber wall and the outer solution (4,5). The magnitude of the pH difference depends on the content of acidic groups, the type of counter ions of the charged groups and also the content and type of ions in the solution.

To prove this during saccharification of steam exploded material, CaOH₂ was added during saccharification of kraft pulp and steam exploded material.

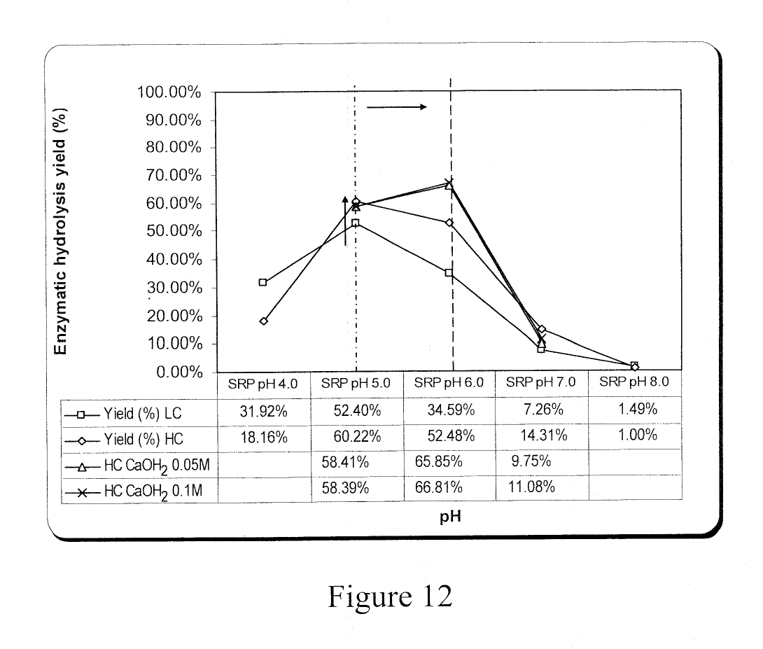

We observe that an increase of ionic strength in the media for kraft pulp saccharification induce a switch on the optimum pH of cellulases used as indicated in FIG. 12. This phenomenon could be explained by a gradient of pH induced by charged moieties in the matrix. The addition of CaOH₂ induces a local pH favorable to move the optimum pH at higher OH— concentration.

FIG. 12 is strikingly similar to FIG. 10 or 11 where PCS was exposed to low and high solids loads. This could suggest that what is happening during high consistency saccharification is an increase in concentration of charged species inducing a gradient of pH in the system.

This effect is caused by the presence of negatively charge groups within the fiber matrix that attract protons (H+). This will create a pH differential between the surrounding liquid and the fiber hence having two pHs, one lower pH at the proximity of the wall and higher in the liquid matrix.

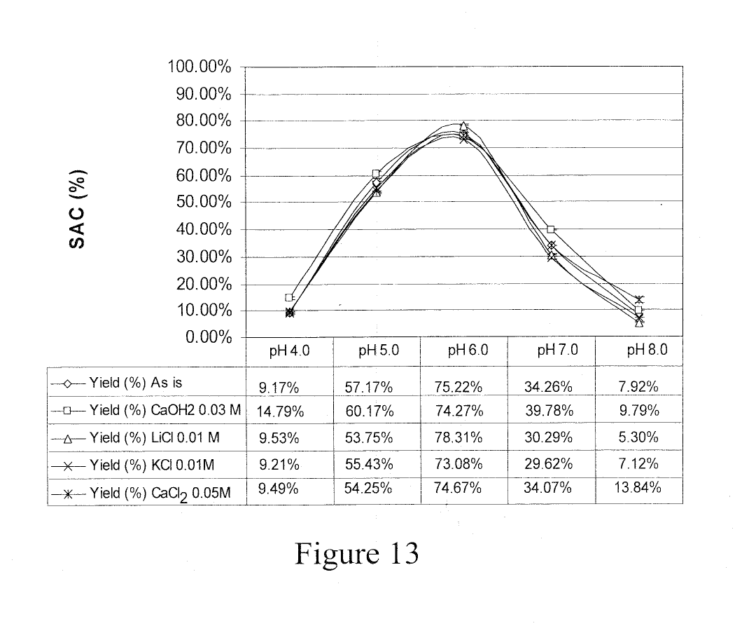

When different ionic strengths were used on PCS, no actual shift on optimal pH was observed as indicated. In FIG. 13 it is shown enzymatic hydrolysis performed at different ionic strength of PCS. The optimum pH at high consistency saccharification is 6 and the addition of different salts did not change this optimum. However when using reference pulp with the addition of CaOH₂ a shift in optimum pH is observed as predicted.

One could infer that during saccharification of kraft pulp when increasing the solids loads and hence less water increases the chances of encounter between fiber and cellulases promoting higher yields, whereas in PCS, the reduced amount of water available would increase the ions concentration inducing a pH switch and that could explain the fact that addition of more salts do not induce a pH switch as it is observed in kraft pulp. The conclusion is that kraft pulp and PCS respond quite differently when treated with cellulases in different environments.

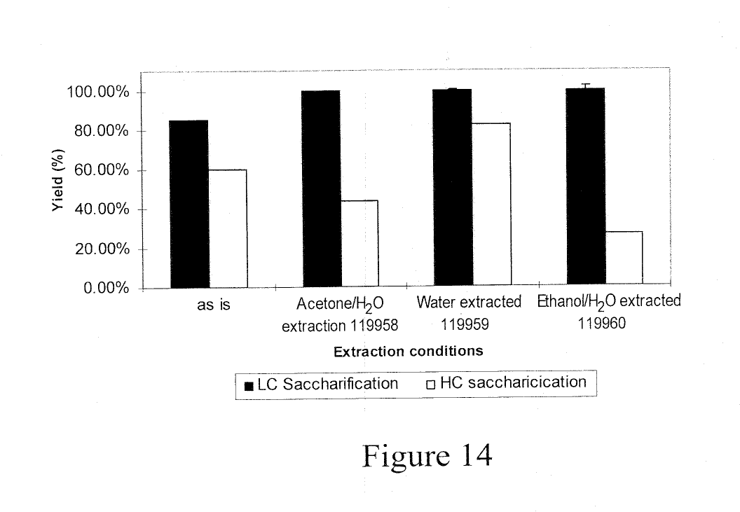

Evidence that could lead to this conclusion as well is by observing the results obtained in a separate experiment using steam exploded corn stover that were submitted to extraction with different solvents as indicated in FIG. 14.

It can be observed that there is a big difference in results whether using low or high consistency. At low consistency, there is no difference in the final results after extraction with different solvents. All of them got 100% conversion. However, using water as a solvent out-performed those pretreated materials that were extracted with acetone or ethanol when using high consistency saccharification. This indicates that water as a protic polar solvent is more efficient removing charged groups or ions as compared with lower dielectric constant such as acetone or ethanol.

This could indicate that the increased concentration of salt/ions at high consistency experiments are possibly causing the need to shift pH in the bulk liquid for optimum enzymes performance.

Based on the above, we conclude that the Donnan phenomena could play an important role during enzymatic hydrolysis of steam exploded corn stover, steam exploded energy cane, and possibly other steam treated materials, especially when performing high consistency saccharification. It has been observed a change in what is always thought to be constant, i.e. cellulases pH optima. Further, an increase of up to 60% in yield could be reached working at higher pH when using higher consistency enzymatic hydrolysis. In this effect, it has been observed that a good pH for conducting the reaction is 5.75 to 6.25. Better results may be obtained if the pH is between 5.9 to 6.1 at the beginning of the reaction and let the system evolve naturally during the rest of the saccharification. In addition, the data shows that the presence of ions in kraft reference pulp promoted a switch on optimum pH of cellulases. Also, removing ions by water extraction induced a better saccharification as compared to organic solvent extractions in steam exploded corn stover.

The experiments and results described above shows that we have been able to achieve a high level of saccharification using high consistency (high solids concentration w/w) cellulosic biomass as feedstock to produce fermentable sugars. The fermentable sugars released from biomass can be used by suitable microorganisms to produce a plurality of target chemicals. The fermentable sugars may be used, for example, as a component of a fermentation broth to make up between 10% to about 100% of the final fermentation medium. These fermentable sugars include 5 carbon sugars (pentose) and 6 carbon sugars (hexose) and may be, for example, glucose, and xylose.

Target chemicals that can be produced by fermentation of the fermentable sugars include, for example, acids, alcohols, alkanes, alkenes, aromatics, aldehydes, ketones, biopolymers, proteins, peptides, amino acids, vitamins, antibiotics, and pharmaceuticals. Alcohols may include, at least, methanol, ethanol, propanol, isopropanol, butanol, ethylene glycol, propanediol, butanediol, glycerol, erythritol, xylitol, and sorbitol. Acids include acetic acid, lactic acid, propionic acid, 3-hydroxypropionic, butyric acid, gluconic acid, itaconic acid, citric acid, succinic acid and levulinic acid. Amino acids include glutamic acid, aspartic acid, methionine, lysine, glycine, arginine, threonine, phenylalanine and tyrosine. Additional target chemicals include methane, ethylene, acetone and industrial enzymes can also be produced.

The fermentation of sugars to target chemicals may be carried out by one or more appropriate microorganisms in single or multistep fermentations. The microorganisms can be, for example, wild type or recombinant bacteria, filamentous fungi and yeast. Such microorganisms include, at least, Escherichia, Zymomonas, Saccharomyces, Candida, Pichia, Streptomyces, Bacillus, Lactobacillus, and Clostridium. As a specific example, the fermentable sugars may be used, for example, for the production of ethanol using yeast, or Z. mobilis or for the production of 1,3-propanediol using E. coli.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

### REFERENCES

1—SSF experimental protocols lignocellulosic biomass hydrolysis and fermentation. Laboratory analytical procedure (LAP) NREL/TP-510-42630. http://www.nrel.gov/biomass/pdfs/42630.pdf. 2—The effect of lignin removal by alkaline peroxide pretreatment on the susceptibility of corn stover to purified cellulolytic and xylanolytic enzymes. Michael J. Selig, Todd B. Vinzant, Michael E. Himmel, Stephen R. Decker. Appl Biochem Biotechnol (2009) 155:397-406. 3—Impact of dilute acid pretreatment conditions and enzyme system on switchgrass hydrolysis. Scott W. Pryor; Nurun Nahar. 2009 ASABE Annual International Meeting. Rene, Nev. Jun. 21, 2009. 4—Impact of the Donnan effect on the action of Xylanases on fibre substrates. Johanna Buchert, Tarja Tamminen, Liisa Viikari. Journal of Biotechnology 57 (1997) 217-222. 5—The role of Trichoderma reesei Xylanases in the bleaching of pine kraft pulp. Johanna Buchert, Marjatta R Anua, Anne Kantelinen, and Liisa Viikari. Appl Microbiology and Biotechnology (1992) 37: 825-829. 6—Fermentation of lignocellulosic hydrolysates. II: inhibition and mechanisms of inhibition. Eva Palmqvist, Barbel Hahn-Hagerdal. Bioresource Technology 74 (2000) 25-33.

We claim:

1. A method for performing high solids saccharification comprising the steps of:
(a) providing a cellulosic biomass;
(b) pretreating the cellulosic biomass in a pretreatment process to produce a pretreated cellulosic biomass;
(c) adjusting said pretreated cellulosic biomass to a solids concentration of 6% to 35% w/w and a starting pH of between 5-7;
(d) hydrolyzing the pretreated biomass with at least one aqueous hydrolyzing liquid comprising at least one enzyme selected from the group consisting of a cellulase, a saccharification enzyme, and a combination thereof for a period of time, to hydrolyze at least a part of the pretreated cellulosic biomass to a cellulosic hydrolysate, said cellulosic hydrolysate comprising a fermentable sugar.

2. The method of claim 1 wherein said pretreating step comprises contacting said cellulosic biomass with at least one aqueous pretreatment fluid to produce a pretreated cellulosic biomass.

3. The method of claim 1 wherein said pretreating step comprises pretreating said cellulosic biomass by steam explosion or advanced steam explosion.

4. The method of claim 3 wherein said advanced steam explosion comprises the steps of:
(b1) pretreating the cellulosic biomass in a first pressurized reactor, wherein the cellulosic biomass undergoes hydrolysis in the first pressurized reactor;
(b2) discharging the cellulosic biomass from the first pressurized reactor to a pressurized sealing device having a first pressurized coupling to a cellulosic biomass discharge port of the first pressurized reactor;
(b3) maintaining a vapor phase in the first pressurized reactor by injecting steam into the first pressurized reactor, wherein the injected steam provides heat energy to the cellulosic biomass in the first pressurized reactor;
(b4) washing the cellulosic biomass in a downstream region of the first pressurized reactor or the pressurized sealing device;
(b5) draining a liquid including dissolved hemi-cellulosic material extracted from the cellulosic biomass from at least one of the first pressurized reactor and the pressurized sealing device;
(b6) discharging the cellulosic biomass from the pressurized sealing device through a second pressurized coupling to a second pressurized reactor, wherein the cellulosic biomass is maintained at a higher pressure in the second pressurized reactor than in the first pressurized reactor;
(b7) in the second pressurized reactor, infusing cells of the cellulosic biomass with steam or water vapor by injecting steam or water vapor into the second pressurized reactor, and
(b8) rapidly releasing a pressure applied to the cellulosic biomass infused with water to cause steam expansion in the cells of the cellulosic biomass and refine the cellulosic biomass to produce a pretreated cellulosic biomass.

5. The method of claim 1 further comprising the step of fermenting the fermentable sugar in a fermentation process utilizing at least one microorganism.

6. The method of claim 5 wherein the at least one microorganism is selected from the group consisting of wild type bacteria, recombinant bacteria, wild type filamentous fungi, recombinant filamentous fungi, wild type yeast, recombinant yeast, and a combination thereof.

7. The method of claim 1 wherein the pretreated cellulosic biomass has a solids concentration of 10% to 35% w/w.

8. The method of claim 1 wherein the pretreated cellulosic biomass has a solids concentration of 15% to 35% w/w.

9. The method of claim 1 wherein the pretreated cellulosic biomass has a solids concentration of equal to or over 18% to 35% w/w.

10. The method of claim 1 wherein said starting pH is between 5.5-6.5.

11. The method of claim 1 wherein said starting pH is between pH 5.7 to pH 6.1.

12. The method of claim 1, wherein the starting pH is maintained between pH 5.7 to pH 6.1 in steps (c) and (d).

13. The method of claim 1, wherein the starting pH is maintained between pH 5.7 to pH 6.1 in step (c) only.

14. The method of claim 1, wherein the starting pH is between pH 5.7 to pH 6.1 in said step C and wherein the pH is decreased in step (d) to a pH of 5.1 to 5.5 over the duration of step (d).

15. The method of claim 1 wherein said period of time in step (d) is between 50 to 200 hours.

16. The method of claim 1 wherein said method achieves a saccharification of said pretreated cellulosic biomass of over 50%.

17. The method of claim 1 wherein said method achieves a saccharification of said pretreated cellulosic biomass of over 60%.

18. The method of claim 1 wherein said method achieves a saccharification of said pretreated cellulosic biomass of over 70%.
